(19) United States
(12) Patent Application Publication
(10) Pub. No.: US 2023/0355495 A1
(43) Pub. Date: Nov. 9, 2023

(54) CONCENTRATED RECONSTITUTABLE CLEANSING COMPOSITIONS FOR PERSONAL CARE

(71) Applicant: GALAXY SURFACTANTS LTD., Navi Mumbai (IN)

(72) Inventors: Nirmal Koshti, Piscataway, NJ (US); Bhagyesh Jagannath Sawant, Kalyan East (IN); Parag Narendra Savla, Bhiwandi (IN)

(21) Appl. No.: 18/311,361

(22) Filed: May 3, 2023

(30) Foreign Application Priority Data
May 5, 2022 (IN) 202221026146

(51) Int. Cl. A61K 8/44; A61Q 19/10; A61Q 5/02; A61K 8/02; A61K 8/46; A61K 8/368; A61K 8/365

(52) U.S. Cl. CPC A61K 8/442; A61Q 19/10; A61Q 5/02; A61K 8/022; A61K 8/466; A61K 8/368; A61K 8/365; A61K 2800/596

(57) ABSTRACT

The invention discloses ‘powder-to-liquid’ hydratable concentrated solid surfactant compositions and a process of making thereof. The compositions are ‘easy-to-transfer’ and ‘easy-to-hydrate’ and upon dilution with water give ‘ready-to-use’ personal cleansing compositions such as hand-wash, body-wash or shampoo. The ‘powder-to-liquid’ compositions are suitable for classical DIY (do-it-yourself) concept enabling the user to create personal cleansing compositions in the same container as and when needed and significantly reducing plastic consumption by the every-day cleansers. The powder compositions of the present invention are free of thickeners (natural gums or synthetic polymers) and the slimy feel. The compositions are biodegradable and are free from toxic antimicrobial preservatives.

### CROSS-REFERENCE TO RELATED APPLICATIONS

This application claims the benefit of priority to Indian Patent Application No. 202221026146 filed May 5, 2022, the disclosure of which is hereby incorporated by reference in its entirety.

### FIELD OF THE INVENTION

The present invention relates to hydratable ‘powder-to-liquid’ cleansing compositions for personal care applications. More particularly, the present invention relates to powder cleansing compositions comprising mixtures of O-acyl methyl isethionate, N-acyl amino acid surfactants as anionic surfactants in a particular ratio and alkylamidopropyl betaines as zwitterionic surfactants, that upon dissolution with water followed by gentle shaking transform unexpectedly into transparent viscous solutions.

### BACKGROUND AND THE PRIOR ART OF THE INVENTION

The concept of converting surfactants-mix in the powder form into ‘ready-to-use’ handwashes by adding it to requisite quantity of water has been around for last couple of years. This is extremely useful and practical concept directed towards drastic reduction of use of plastic by encouraging consumers to ‘do-it-by-themselves’ the cleansing compositions for personal hygiene. In addition to restricting plastic consumption, a significant saving of energy is achieved in terms of transportation when consumers use water-less, concentrated compositions in paper bags/sachets and reconstitute it by simply adding to definite amount of water and shaking gently in a refill container to get the final personal or household cleansing formulation. The examples of powder hand-wash products in the market are Lifebuoy powder to liquid handwash by Unilever, ‘Mr. Magic’ handwash by Godrej Consumer products, India and Savlon antibacterial powder hand wash by ITC Ltd, India. Companies in Scandinavia, ‘All Matters’ (Denmark) and Superkons and Forgo (both from Sweden) are actively promoting this sustainability concept of reconstitutable ‘powder-to-liquid’ for handwashes. ‘Earth Buddy’ in India is promoting the same concept for handwash, dishwash and hard surface cleaners.

The Powder-to-liquid' concept for personal care washes is reported in 2004 (Lambino, Rehydratable Personal Care Compositions, EP 1574203A), however, this concept seemed to have become relevant due to pandemic that forced the human population to take care of hand hygiene more frequently compared to normal times. Now, with global warming getting out-of-control, every effort by a global citizen/consumer is welcome that contributes towards achieving the tall goals that are designed to stop and reverse the process of global warming. The concept of concentrated and reconstitutable compositions address and contribute towards saving on energy, significant reduction in non-degradable material, burning less fuel for road transportation etc.

EP 1574203A discloses dried rehydratable personal care compositions with significant inclusion of gelling agents along with protic hydroxylic solvents like glycerol, propylene glycol or dipropylene glycol to enhance the ease of hydration. EP 1574203A teaches the use of natural polymers such as Guar, Carrageenan, Sod. alginate, Pectin, Starch, Xanthan gum, Dextran, Karaya gum, Tragacanth gum, Gum Arabic, Locust bean gum, Ghatti gum, Agar, and Gellan gum; animal derived Casein and Gelatine etc., modified natural polymers such as hydroxypropyl cellulose, hydroxyethyl cellulose, hydroxy propyl guar etc.) and synthetic polymers such as acrylates and acrylamides or alkylene oxide polymers as gelling agents.

EP '203A deploys protic solvents like glycol (1 to 20% by weight of the composition) as a part of composition. It is necessary to dissolve or disperse the gums in hydroalcoholic solution since gums are hard-to-disperse when they gel in the presence of water.

Use of microparticulate hydrocolloids (Agar, Carrageenan, Guar gum, Starch, Cellulose etc.) for hydration and gelling effect is reported with surfactant, detergent, enzyme, a food additive, flavouring agent, with medicinal molecule, pharmaceutical active or pesticide and herbicide. Another patent application by Thiewes et al. (US 2013/0267612 A1) disclosed personal care composition after reconstituting powder mix containing water-soluble starch and dehydro-xanthan gum. It claims ‘instant’ powder for aqueous solution.

There are a few products in the market that offer the surfactants blends in small paper bags that can be emptied in a refill container with dispensing pump, adding water up to the mark and allowing the mixture of water and surfactants to sit for a few hours after gently shaking the bottle. The container with a pump is a refill container that consumer uses as and when the refill composition is needed. One such product launched by Godrej consumer products in India is ‘Mr. MagicHandwash's www.godrejprotekt.com/personal-hygiene/mr—magic-hand-wash?msclkid=9ef5eff5a68allec90d0094e317dbe79). The powder composition in the paper sachet is made up of sodium lauryl sulphate and disodium lauryl sulphosuccinate as anionic surfactants and guar gum as gelling/thickening agent. It is preserved by parabens (known for its toxic, endocrine disruptor) and ETDA (known for ecotoxicity concern, slow to biodegrade and supports algal growth). The patent application IN 201821030525 discloses the use of gelling agents/gums like guar, cellulose and other natural gums. like Guar gum, Xanthan gum, Cellulose and its derivatives like hydroxy propyl methyl cellulose, and carboxy methyl cellulose etc. without hydroxylic solvents. The reconstituting surfactants blends with gums in powder form suffers from two major disadvantages. Firstly, the re-constitution process takes a long time as hydration of polymers takes time since water has to penetrate the polymeric network to reach the core of the particle and secondly, despite long hydration times, the final preparation is not a smooth and uniform due the presence of small jelly like particles that cannot be dispersed or broken down by simple hand-shaking of a refill-container. The reconstituted handwash formed is not transparent due to limited solubility of gums in water. This limitation can be easily seen in the products (above mentioned reconstitutable handwashes) in the market. Also, the instability (limited stability) of the reconstituted products is observed in the market products on standing. The reconstituted product as made as directed, doesn't seem to have long term stability at ambient temperature and phase separation is observed. The most likely reason of this anticipated problem is the re-agglomeration of the gum particles in reconstituted products.

Dierassi et al. (U.S. Pat. No. 4,330,438) teaches ‘powder shampoo’ concentrate with anionic surfactants (sodium lauryl sulphate and C₁₄-C₁₆ alpha olein sulphonate) and hydroxy propyl guar gum. This non-ionic guar gum derivative is claimed to afford the desired viscosity and the retained film on the hair is said to condition the hair. The disclosure doesn't mention about transparency of the final formulations. Further, the inclusion of harsh surfactant (sodium lauryl sulphate) as a primary surfactant in the compositions and its harsh effects of SLS (stripping of lipids and proteins from skin and hair) were not appreciated.

Recently, ‘All Matters’, the company from Denmark introduced ‘powder-to-foam’ body wash which is based on a gum (xanthan gum) and anionic surfactants like sodium coco sulphate and sodium cocoyl glutamate. Another Swedish company, Forgo, is marketing both hand wash and body wash that are ‘sulphate-free’ using sodium cocoyl glutamate, inulin and a-glucan oligosaccharide. The latter two ingredients act as gelling and thickening agents in addition to being prebiotics.

It is understood from the aforesaid that the recently launched products are based on the old concept of using gums (natural polymers, Lambino, ‘Rehydratable Personal Care Compositions’, EP 1574203A) and focused on this worthy and sustainable trend of ‘powder-to-liquid’, ‘easy-to-reconstitute’ format for hand-washes, body-washes and hair-washes. In most cases sodium cocosulphate (since it is available in powder form) is used which is known for its harshness (strong ionic interaction with proteins of corneocytes) and drying effect (stripping of lipids of stratum corneum). The major constraint of this ‘powder-to-liquid’ format products present in the market is that one cannot use emollients and moisturizers that are often liquids in nature and hence unsuitable for this dry formulation format. Also, another big drawback or the limitation of these dry powder formulations is the non-availability of a natural gum type thickening or a gelling agent that would get dispersed easily without having to wait fora long time (soaking time) as is the case with most of the gums and polymers. The dispersion of these either natural or synthetic polymers doesn't happen with ease, by simple shaking of the container. The gentle manual shaking of the container is woefully inadequate to shear the small jelly like lumps that are formed and to disperse uniformly in this typical ‘do-it-yourself’ concept. Also, with gums, either natural or semi natural-semi synthetic, it is not possible to create transparent formulations. This is a serious limitation. In case of synthetic thickeners (polyacrylic acid type), the pH of composition needs to be in the alkaline zone for thickening the surfactant solution, thus creating a serious disadvantage for personal care formulations with pH way above the skin pH of 5.5. It should be noted that almost all synthetic polymers are not readily biodegradable. The other concern while using the natural polymers (carbohydrates and proteins) in water is the need for strong antimicrobials for preservation. Use of toxic antimicrobials have deleterious effect on the ecology when personal cleansers go back to Mother Nature after the usage. Further, the use of gums and natural polymer or synthetic polymer in the significant levels compromises the active content of surfactants and hence result into poor cleansing experience.

The personal care cleansers like handwash, bodywash and shampoo have been conventionally viscous, and transparent. The transparency of these formulations is often associated with purity and aesthetics. Natural gums and their derivatives not only don't form the quick viscous end formulation but both transparency and smoothness of gel are compromised. Synthetic polymers need higher pH for them to gel well and they are not readily biodegradable due to macromolecular nature and due to cross-linking. These limitations are easily seen in the offerings in the market. In addition to the compromised aesthetics in ‘powder-to-liquid’ compositions, the biggest performance limitation coming from the use of thickening agents is slimy feel imparted by the gummy and polymeric thickeners to the end-formulations. Consumers have to use plenty of water to get ‘squeaky clean feel’ after rinsing off the wash preparation.

In view of these serious limitations, it is important to have a ‘powder-to-liquid’ format with a thickening agent that would quickly work with simple agitation (few shakes to the refill-container) to give decent viscosity, transparency at biological pH of skin without compromising on biodegradability.

U.S. Pat. No. 11,045,404 reports self-thickening surfactant compositions by mixing sodium N-acyl glutamate or its acid form (acyl group is either cocoyl or lauroyl) with an amphoteric/zwitterionic surfactant like alkyl betaine or alkyl amido propylbetaine or alkyl amidopropyl hydroxy sultaine (alkyl group of betaines is either C₁₂ or C_{8-18,} coco) and water to be self-thickening. US'404 teaches about difficulty of building viscosity using amino acid surfactants (column 1, line 58-62) by commonest methods like addition of salts or other common and economicalsurfactants like coco amidopropyl betaine. (for example, thickening of solutions of sodium laureth sulphate with coco amidopropyl betaine and/or by addition of common salt),It is also pertinent to mention here that the composition becomes self-thickening at one specific pH value and not over a range of pH. This criticality of extremely narrow pH range is evident from the examples cited. To practice this art on an industrial scale is tough since it is very difficult to get one fixed number of pH particularly when viscous material is being stirred. A small deviation from that value results in abnormal and drastic changes in viscosity. Hence, it is not industrially viable.

Unexpectedly, the inventors of this patent application have found surfactant combinations in powder form that on dilution with water result in viscous (isotropic and lamellar) solutions that are transparent with pH between 5.0 to 6.0 (skin pH),It avoids the use of any synthetic or natural polymers for gelling or thickening effect and all the problems associate with the stability, aesthetics (hazy and slimy) and ease-of-operation. It eliminates need for strong preservation and provides superlative performance at the biological pH of skin without having to use harsh surfactants like SLS.

### OBJECTIVES OF THE INVENTION

It is an objective of the present invention is to create a user-friendly ‘powder-to-liquid’ compositions for personal care application without the drawbacks of the prior art.

It is another objective of the present invention is to create a user-friendly ‘powder-to-liquid’ compositions that would be easy to reconstitute (ideal for ‘do-it-yourself’ concept) and would be stable infinitely without any physical separation for personal care composition.

It is a further objective is to create ‘powder-to-liquid’ compositions which upon dissolution in water would thicken without aid of natural or synthetic polymeric gelling or thickening agent.

It is yet another objective of the present invention is to create ‘powder-to-liquid’ compositions which upon dissolution in water would afford the personal care formulations like hand wash, bodywash and shampoo without any slimy feel during the use as well as after the use.

It is also an objective of the present invention is to create ‘powder-to-liquid’ compositions which upon dissolution in water would afford the personal care formulations like hand wash, bodywash and shampoo at biological pH range of skin 5.0 to 6.0.

It is another objective of the present invention is to create ‘powder-to-liquid’ compositions which upon dissolution in water would afford the personal care formulations like hand wash, bodywash and shampoo without harsh surfactants like alkyl sulphate or ether sulphates.

It is another objective of the present invention is to create ‘powder-to-liquid’ compositions which upon dissolution in water would afford the personal care formulations like hand wash, bodywash and shampoo with superlative performance of foam and lather with mild surfactants that are gentle to skin and hair.

It is yet another objective of the present invention is to create ‘powder-to-liquid’ compositions which upon dissolution in water would afford the personal care formulations like hand wash, bodywash and shampoo with absolute transparency and good aesthetics.

It is yet another objective of the present invention is to create concentrated ‘powder-to-liquid’ compositions with minimum 80% of surfactants and minimum formulation aids like stabilizing or thickening agents which upon dissolution in water would afford the personal care formulations. This allows dilution of powders up to 10 times giving maximum benefit in terms of plastic waste. The concentrated formulations all wide scope for creating the range of personal care cleansing that includes facewash, handwash, bodywash and shampoo using varying concentration of powders.

### SUMMARY OF THE INVENTION

Accordingly, the present invention provides a reconstitutable concentrated powder composition, said composition comprising:
a. 50 to 70%, by weight of total powder composition, a blend of O-acyl methyl isethionate and N-acyl amino acid surfactants;
b. 25 to 40%, by weight of total powder composition, an alkylamidopropyl betaine;
c. 2.0 to 6.0%, by weight of total powder composition, an antimicrobial preservative; and
d. Optionally, 1.0 to 10.0%, by weight of total powder composition, one or more cosmetically or pharmaceutically acceptable auxiliary,
   wherein said blend of O-acyl methyl isethionate and N-acyl amino acid surfactants are prepared sequentially from same fatty acid chloride;
   said O-acyl methyl isethionate and N-acyl amino acid surfactants being present at the ratio of between 2:1 and 1:2; and
   said reconstitutable concentrated powder composition when diluted by 4 to 12 times by weight of said powder, with water affords a transparent liquid formulation with pH ranging from 5.0 to 6.0 and viscosity ranging from 1,000 to 15,000 cps at 25° C.

The above-described features and the advantages of the present disclosures will be appreciated and understood by those skilled in the art from the ‘detailed description’ and the ‘claims’.

### DETAILED DESCRIPTION OF THE INVENTION

As mentioned in the background section, ‘DIY’ concept for everyday personal cleansing (facewash, handwash, bodywash and shampoo) is important for several consumers worldwide who are very conscious about the global warming and the precarious condition the world finds itself as on today. Many consumers are aware of the damage caused by excessive use of nonbiodegradable materials and their responsibility towards addressing this issue. They are keen on contributing individually towards saving of the planet. The global consciousness is very high about the fact that serious measures need to be taken to reverse the damage done by thoughtless excessive use of plastic. In addition to avoiding use of plastic, this DIY concept also contributes towards significant saving of energy that is spent in packing the dilute products (containing a large amount of water) and burning of fossil fuel for transporting the dilute compositions.

The ‘Powder-to-liquid’ reconstitutable compositions of the present invention for personal cleansing offer stable, cosmetically acceptable aesthetics. These compositions are ‘easy-to-reconstitute’ because, unlike the prior art, these are made without natural gums or any other synthetic polymeric thickeners which normally result in sticky and stringy feel of the end-composition. Another serious limitation is the time taken by these macromolecules (thickeners) for hydration and swelling of particles forming a heterogenous structural arrangement with surfactant molecules. Surfactants are amphiphilic molecules and the thickeners (natural gums or synthetic polymer) are hydrophilic but their polymeric nature and the crosslinked polymeric chains make them difficult to wet compared to small molecules. The gentle shaking of refill container, that is essence of the ‘DIY’ concept, doesn't result in a homogenous phase with long term stability of the resultant composition. This is a serious limitation of the prior art. High shear mixing of swollen polymer particles (which are often crosslinked) is not possible in ‘DIY’ concept where refill container is shaken gently by the consumer. Another significant disadvantage of the reconstituted compositions with natural gums do not give in consumer desired in-use performance in terms of lather and foam. The gums make the compositions very slimy and not only they suppress the generation of lather but they also consume a lot water for removing the slimy composition from the surface of the hands or body. The gum-based compositions are difficult to rinse-off and consume large amount of water. This is exactly contrary to the purpose of sustainability and water conservation. These limitations, 1) difficulty in reconstituting by gentle shaking of the ‘refill container’, 2) stability of resultant composition, 3) decent aesthetics (transparency, smoothness of the texture) and 4) amount water required for rinsing after the application, have been addressed by the present invention by unexpectedly creating micellar or lamellar arrangement of specific surfactants for personal wash formulations. This has been achieved at the desired biological pH of skin avoiding all harsh soaps and sulphated surfactants.

### The Anionic Surfactants

The anionic surfactants used in the present invention are combinations of O-acyl methyl isethionate (Formula I) and N-acyl amino acid surfactants. These are produced according to the procedure described in U.S. Pat. No. 9,308,156. The O-acyl methyl isethionates employed in creating combinations such as sodium lauroyl methyl isethionate (CAS No 928663-45-0) and sodium cocoyl methyl isethionate (CAS No 2244880-58-6). O-Acyl methyl isethionates can be made from single alkyl chain (C₁₂, lauroyl) or they can be mixtures of more than one alkyl chains. Most common alkyl chains derived from vegetable fatty acids (from vegetable oils) are of C₈ to C₁₈. However unsaturated fatty acids like undecylenic acid or oleic acid or linoleic acid or linolenic acid can also be employed. Similarly, vegetable fatty acids (behenic acid or arachidic acid etc.) with carbon higher than 18 can be employed for making O-acyl methyl isethionates of the present invention.

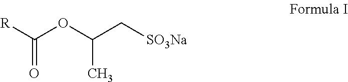

Wherein, R=C₁₁ or C7 to C₁₇.

The N-acyl aminoacid surfactants are deployed in creating combinations as demonstrated in Table 1. N-acyl amino acid surfactants along with O-acyl methyl isethionates are N-acyl sarcosinates, N-acyl glutamates, N-acyl glycinates and N-acyl taurates creating various novel reconstitutable cleansing compositions for personal care. The acyl group can be typically C₈ to C₁₈ since these are derived from even numbered fatty acids from vegetable oil. Unsaturation in the alkyl chain is common in naturally derived fatty acids of C₁₈ or higher. The same fatty acid chloride is used for the synthesis of both families of surfactants, acyl isethionates and acyl amino acids salts and hence the alkyl chain distribution remains the same in terms of number of carbons and unsaturation. of O-acyl isethionates and N-acyl amino acids salts is the same.

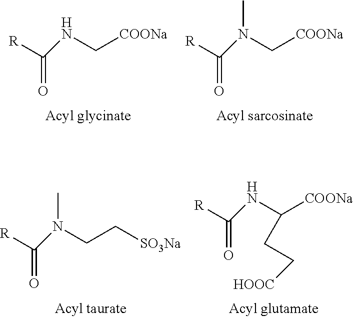

The ‘sulphate-free’ surfactants are getting increasingly popular in personal cleansing due to their mildness on the skin (towards the lipids and proteins of uppermost layer of the skin, the stratum corneum) and hair. In addition, they do provide good foam and lather which is a consumer desired attribute of a personal cleanser. In addition to luxurious lather, outstanding rinsability (in-use performance), the soft after-feel (post-use performance) are also consumer desired attributes.

The combination O-acyl methyl isethionate and N-acyl amino acid surfactant is made by a pseudo one pot synthesis. The process of creating combination involves two steps, A) making of fatty acid chloride and B) making both surfactants using same fatty acid chloride in a pseudo one pot synthesis. The process involving these two steps is totally green. Both families of surfactants are synthesized by a common starting material of fatty acid chloride which is synthesized using biodegradable heterogenous surfactant catalyst for chlorination and whole process meets all twelve principles of ‘Green Chemistry’ (U.S. Pat. No. 9,187,407). Fatty acid chloride is reacted with powder form of sodium methyl isethionate in anhydrous conditions and the hydrochloride gas generated is scrubbed. The excess fatty acid chloride along with the sodium O-acyl methyl isethionate produced is then reacted with an amino acid in the presence of a base in aqueous conditions to create N-acyl amino acid surfactant. This results in aqueous solution of both surfactants (isethionates and amino acid surfactants) in the chosen molar ratio (U.S. Pat. No. 9,308,156 B2).

The aqueous solution containing both surfactants is then spray dried to get the mixtures of O-acyl methyl isethionate and N-acyl amino acid surfactants as given in the Table 1 below. The detailed representative procedure is given in the experimental section covering TLMI 11, TLMI 21, GLMI 11 and TCMI 11.

In mild surfactants combinations described herein are created with one O-acyl methyl isethionate with one N-acyl amino acid surfactant. However, more than one amino acid can be deployed to create combinations more than two surfactants, for example, a combination of sodium O-lauroyl methyl isethionate with N-lauroyl glutamate and N-lauroyl methyl taurate etc.

TABLE 1
Spray dried mixtures of O-acyl methyl isethionate
and N-acyl amino acid surfactants.

| | | O-Acyl methyl | |
|---|---|---|---|
| Code | N-Acyl amino acid surfactant | isethionate | Ratio |
| GLMI 11 | sodium lauroyl glutamate | sodium lauroyl | 1:1 |
| | | methyl isethionate | |
| GLMI 21 | sodium lauroyl glutamate | sodium lauroyl | 2:1 |
| | | methyl isethionate | |
| TLMI 11 | Sodium lauroyl methyl taurate | sodium lauroyl | 1:1 |
| | | methyl isethionate | |
| TLMI 21 | Sodium lauroyl methyl taurate | sodium lauroyl | 2:1 |
| | | methyl isethionate | |
| TCMI 11 | Sodium cocoyl methyl taurate | sodium cocoyl | 1:1 |
| | | methyl isethionate | |
| SLMI 11 | sodium lauroyl sarcosinate | sodium lauroyl | 1:1 |
| | | methyl isethionate | |
| GMI 11 | Sodium lauroyl glycinate | sodium lauroyl | 1:1 |
| | | methyl isethionate | |
| TLMI 41 | Sodium lauroyl methyl taurate | sodium lauroyl | 4:1 |
| | | methyl isethionate | |
| TLMI 14 | Sodium lauroyl methyl taurate | sodium lauroyl | 1:4 |
| | | methyl isethionate | |
| GLMI 14 | Sodium lauroyl Glutamate | sodium lauroyl | 4:1 |
| | | methyl isethionate | |
| GLMI 14 | Sodium lauroyl Glutamate | sodium lauroyl | 1:4 |
| | | methyl isethionate | |

The typical process of synthesizing combinations of these two families of surfactants from the same fatty acid chloride as described in U.S. Pat. No. 9,308,156B2 and is illustrated with GLMI 11 (sodium lauroyl glutamate and sodium lauroyl methyl isethionate in the molar ratio of 1:1), TLMI 11 (sodium lauroyl methyl taurate and sodium lauroyl methyl isethionate in the molar ratio of 1:1), TLMI 21 (sodium lauroylmethyl taurate and sodium lauroyl methyl isethionate in the molar ratio of 2:1) and TCMI 11(sodium lauroyl methyl taurate and sodium lauroyl methyl isethionate in the molar ratio of 1:1)in the experimental section. All the combinations described in the Table 1 are produced as aqueous solutions (30-40% solids content) and on spray drying all the liquids are converted into free-flowing powder with particle size typically less than 250 microns and moisture content of 1 to 2%. Besides moisture content the dried powders contain sodium chloride around fifteen per cent. The active content of the powders is calculated by subtracting moisture content and salt content from 100.

The spray dried powders of surfactant combinations of Table 1 are then blended with powder form of zwitterionic surfactants and other ingredients in powder form as described in Table 4. The anionic surfactant combinations of Table 1 are deployed at 50 to 60% by weight of the total ‘powder-to-liquid’ compositions of Table 4.

### Zwitterionic Surfactants

The zwitterionic surfactants that are used in the present invention along with the combinations of anionic surfactants (Table 1) described above, are alkyl amino propyl betaines, namely, lauramidopropyl betaine (CAS No 4292-10-8) and cocamidopropyl betaine (CAS No 61789-40-0). Typically, these zwitterionics are commercially available as aqueous solutions by several manufacturers globally. The commercial aqueous composition typically has 30% betaine with about 5 to 6% sodium chloride. The commercially available aqueous solutions are spray dried. The commercial aqueous products afford free flowing spray dried powder with the active zwitterionic content around 80-85% and the balance being mainly sodium chloride. The spray dried powders of alkylamidopropyl betaines are blended with the anionic surfactants' combinations of Table 1 to get the ‘powder-to-liquid’ compositions of Table 4.

Alkylamidopropyl betaines, the zwitterionic surfactants, are deployed at 30 to 40% by weight of the total ‘powder-to-liquid’ compositions of Table 4.

### Antimicrobial Preservatives

The antimicrobial preservatives of the present invention are organic acids. Antimicrobial organic acids work efficiently at the pH range of 5.0 to 6.0 of the compositions of the present invention after reconstituting the powders. Benzoic acid (CAS No 65-85-0, MP 122° C.), sorbic acid (CAS no 110-44-1, MP134° C.) and dehydroacetic acid (CAS No 520-45-6, MP 109° C.) are well accepted personal care and food products preservatives with a strong action against fungi. The other two organic acids employed in the compositions described in the Table 4 are undecylenoyl glycine (CAS No 54301-26-7, MP 103° C.) and capryloyl glycine (CAS No 14246-53-8, MP 102° C.). All above organic acids are suitable for the present invention since a broad spectrum of antimicrobial protection can be created at the biological pH of skin. The organic acids are relatively ‘non-toxic’ compared to parabens, and halogenated antimicrobials or formaldehyde donors etc. The acid forms are good solids with high melting points and hence all can be easily accommodated in solid form of ‘powder-to-liquid’ reconstitutable compositions of the patent application. The antimicrobials mentioned above are in fine powder form to ease of blending with the powder zwitterionic and the anionic surfactants.

The antimicrobial preservatives of organic acid type are deployed at 2.0 to 6.0% by weight of the total ‘powder-to-liquid’ compositions as demonstrated in Table 4. These can be used alone or in combination of more than one organic acid preservatives to exploit the synergy of antimicrobial action. The other in liquid form and relatively non-toxic antimicrobial like phenoxy ethanol can be used along with the solid antimicrobials in the compositions of Table 4.

### The Optional Additives

The optional skin or hair benefit agents are limited only to the extent that they are capable of being topically applied and are suitable to dissolve in the end composition that are reconstituted from the powders. They include amino acids like arginine, valine, histidine, glutamic acid or glycine. Vitamins can be selected from Vitamin B2, Vitamin B6, Niacinamide or Ascorbic acid and its derivatives. Hyaluronic acid or pyrrolidine carboxylic acid (PCA). Dried water-extracts of herbs or plants from traditional medicine can be the part of optional benefit agents and these include, Aloe vera, cucumber, turmeric, liquorice, Rosemary, Sage etc. Essential oils can be part of the powder compositions. Examples are Neem oil, Jasmine oil, Lavender oil or Tea tree oil. Other benefit agents that can be employed optionally, are oil-soluble vitamins (A, D and E), omega-3-fatty acids, ceramides. Other benefit agents based on carbohydrates or proteins can be added for other skin and hair benefit though these are not needed for modifying the rheology. Anti-acne agents or antidandruff, particularly, those in keeping in line with sustainable chemistry and renewable manufacture like undecylenoyl glycine or capryloyl glycine can be added. These lipidated glycines are derma purifier and are effective against body odour causing microbes (microbiome of axilla, the major genera are Staphylococci, Corynebacteria and Cutibacteria). The lipidated glycines have significant activity against Malassezia yeast which is the main yeast of human microbiome. For conditioning effect, particularly for hair, the cationic conditioners that are available as powder can be used, for example, Guar hydroxy propyl trimethyl ammonium chloride. Equally popular behenamidopropyl dimethyl amine (CAS no, 60270-33-9, BAPDA) or stearamidopropyl dimethyl amine (CAS no, 7651-02-7) can be incorporated, in the powder form, into the concentrated surfactant blends of the present invention. Anti-caking agents are also optional since in some cases where moisture content of the powder mix needs to be tackled then silica compounds can be used. The colour and fragrances are optional and some of the compositions of Table 4 have been made with both water soluble and oil-soluble fragrances. The optional additives are present in the range of 1.0 to 10% by weight of total powder composition that is then reconstituted to get the liquid composition for personal cleansing. The optional additives may include hair actives, skin actives, dry herbal extract, chelating agents (sodium gluconate and citric acid), salts (sodium chloride), vitamins, moisturizing agents, soothing agents, nourishing agents, conditioners, colors and fragrances.

### Preparation of ‘Powder-to-Liquid’ Compositions and Reconstituting the Same to Create Personal Cleansing Formulations

The anionic surfactants of Table 1 are prepared as given in the experimental section. The aqueous solutions of the combinations of sodium O-acyl methyl isethionate and N-acyl amino acid salts are spray dried to get fine powder with all the particles having size of less than 300 micron. Using same spray drying conditions, alkyl amidopropyl betaines are dried to afford free flowing powder of zwitterionic surfactants. These two classes of surfactants are blended together with powdered antimicrobial preservatives and optional ingredients (Table 4) using a low shear powder blender.

Usual powder blending technique work very well (V-blender or Nauta type conical blender with a screw type agitation.

GLMI 11 (Table 1) is a blend of sodium lauroyl methyl isethionate and sodium lauroyl glutamate in the ratio of 1:1. GLMI 11 is blended with zwitterionic surfactants and preservative and other ingredients (Ex 1 and 2 of Table 4). This blended composition is then reconstituted with water as described in the Experimental section with simple hand shaking the container. At 16% concentration in water, the liquid compositions of Ex. 1 and Ex. 2 are transparent with pH close to skin pH and with viscosities of around 2500 to 3000 cps. The molar ratio of two anionic surfactants is 2:1 in GLMI 21 (2 moles of sodium lauroyl glutamate and 1 mole of sodium lauroyl methyl isethionate). The blend of Ex. 3 using GLMI 21, upon reconstitution gives all the desired attributes with slightly lower viscosity compared to examples with GLMI 11. However, GLMI21 based blend (Ex. 11) with 21% solids content (approx. 5 times dilution) results in viscosity of 14,500 cps.

Examples 4, 5, 6 and 7 of Table 4 are based on TLMI 11 (sodium lauroyl methyl taurate and sodium lauroyl methyl isethionate in 1:1 molar ratio). Examples 5, 6,& 7 are made with TLIM 11 and Galguard LipoG (which is a blend of undecylenoyl glycine and capryloyl glycine in molar ratio of 1: 1) at 2.5% w/w. Blends of Ex. 5 & 6 are reconstituted to liquids with 21% solids content with viscosities in the range of 4000 to 6000 cps. Lowering the solids content (18%) results in lowering of viscosity (2100 cps) as demonstrated in Ex. 7. By changing the relative ratio of lauroyl taurate to lauroyl methyl isethionate as in TLMI 21, the blend of Ex. 9 (Table 4) after reconstituting into liquid with 21% solids w/w has viscosity of 10,000 cps. In TCMI 11 (Table 1), the same pair of anionic surfactants category with 1:1 molar ratio is combined, however, lauroyl chain has been replaced by cocoyl group. Example 10 of the Table 4 reports a blend that upon reconstitution at 21% solids (roughly five times dilution) resulted in clear transparent liquid with viscosity of 6050 cps.

Examples 12 and 13 are fully formulated blends with water-soluble fragrance (CPL Aromas, Mumbai, AR 634412) at 1.5% level. Upon reconstituting by six times dilution (approx. 16% solids content) with water, the resultant compositions have viscosities in the zone of 6000 to 7000 cps. Addition of fragrance doesn't change any physical characteristics of the composition including bulk density.

Examples 14 and 15 are fully formulated with oil-soluble fragrance (Sonarex 9090409, Sonarex High Tech Aromatics PVT.Ltd.) at 1.5% level. Upon reconstituting by six times dilution (approx. 16% solids content) with water, the resultant compositions have viscosities in the zone of 6000 to 8000 cps. Example 16 of Table 4, consist of TLMI 21 based powder mix, was made with the popular hair conditioner Guar hydroxypropyl trimethyl ammonium chloride (GHTC) at 1.0%. Reconstituting the powder to liquid by six times dilution is easy, only difference is the liquid constitution has light haze due to dispersion of GHTC. Example 16a, consists of TMLI 11 based powder mix, was made with other hair conditioning agent, behenamidopropyl dimethyl amine (BAPDA) at 3.5%.

The preferred ratio of the blends of O-acyl methyl isethionate and N-acyl amino acid surfactant is 2:1 to 1:2. To get best synergistic performance both surfactants should be present in desired ratio.

Lowering any one of the two surfactants (either isethionate or amino acid surfactants) to extremely low level in a combination, results in the loss of the advantage generated by the synergy of isethionate type surfactant and acylated amino acid surfactant when combined together. This is further demonstrated by Examples 17, 18, and 19 of Table 4a, showing that the formulations made from dry products of TMLI (Table 2) where the ratio is 4:1 and 1:4, result into composition (after reconstitution) of poor viscosity even at high solids content of 22%. Comparative Examples 17-19 are illustrated with a combination of sodium lauroyl methyl isethionate and sodium lauroyl methyl taurate (Table 1 and 2) Similarly, examples 20, 21, and 22 illustrate (Table 4a) the above point with sodium lauroyl methyl isethionate and sodium lauroyl glutamate at ratio of 4:1 and 1:4 (Table 1 and 2). Typically, face wash or baby wash formulations are with lower concentrations of cleansing surfactants whereas shampoo has the highest surfactant concentrations. Example 17 with low concentration (solids content of 11%) and Examples 18 and 19 with high concentration (solids content of 22%) doesn't generate enough viscosity (<1000 cps) if the concentration of the either of the two is lowered beyond a point. Hence, the preferred ratio of the blends of O-acyl methyl isethionate and N-acyl amino acid surfactant is 2:1 to 1:2.

### Advantages

1) The present invention addresses the drawbacks of the prior art. It is directed towards conscious consumers who are looking for opportunities to reduce the environmental waste, particularly the plastic waste. The serious limitations of the ‘powder-to-liquid’ prior art are
   a) difficulty to hydrate the powder due to longer soaking time needed for the thickeners in the compositions that are polymericin nature, b) difficulty in dissolving the powder when added to water by moderate shear generated by gentle shaking of the container and resulting into inhomogeneous end-formulation with seriously compromised transparency and general aesthetics c) very narrow working range of pH to maintain the viscosities.
2) The ‘powder-to-liquid’ composition of the present invention is without any structuring or gelling or thickening agent and hence it offers extreme ease in hydrating and dissolving in water and achieving the desired viscosity, transparency for micellar or lamellar end-formulations. The ‘ease-of-making’ with desired ‘aesthetics’ the end-formulation by the consumers is fundamental to ‘DIY’ concept.
3) The ‘powder-to-liquid’ composition of the present invention is without any stabilizing agent or thickening agent or gelling agent or hydratable hydrocolloid of natural or synthetic origin. The closest prior art talks about use of such polymers or gums as high as 20 to 30% (IN 201821030525). This adds to the cost of formulation significantly since polysaccharide-based gums (guar, xanthan or carrageenan or cellulosic derivatives or totally synthetic, acrylate based carbomers, are extremely expensive and also their deployment at such high % level adds significantly to the environment load. These relatively expensive ingredients are purely formulation aids and do not possess any surfactant action that would contribute to cleansing of skin and hair.
4) The ‘powder-to-liquid’ compositions of the present invention are real concentrated cleansing compositions with no formulation-aids. The anionic surfactants and zwitterionic surfactants form 90% of the composition. This is possible due to the surfactant systems of these compositions that give the desired stability, structure and performance in terms of gentle cleansing with superlative consumer desired attributes of foam and lather.
5) The ‘powder-to-liquid’ compositions of the present invention are ‘sulphate-free’. Soap (high alkaline pH) and harsh protein denaturing sulphated surfactants are totally avoided. The compositions of the present invention, after reconstituting with water result in liquid compositions at skin pH (5 to 6). Non-disturbance of skin pH is important for the effective functioning of skin microbiome.
6) The transparent end-formulations (liquids obtained after reconstituting the powder) of the present invention exhibit good sensorial properties with excellent foam and lather performance.
7) The transparent end-formulations (liquids obtained after reconstituting the powder) of the present invention are free from any slimy feel during or after use and impart ‘squeaky clean’ feel post rising. This is contrast to the ‘Powder-to-liquid’ products available in market that are based on natural gums or synthetic polymers which are extremely slimy and often require excessive amount of water for rinsing-off after the application.
8) It uses blends of Acyl methyl isethionate and N-acyl amino acid surfactants that are made by fatty acid chloride route using green technology (U.S. Pat. Nos. 9,187,407 B2 and 9,308,156 B2).
9) The ‘powder-to-liquid’ compositions of the present invention use N-acyl glycines as skin benefit agents that are made using green technology (U.S. Pat. No. 9,187,407).
10) The ‘powder-to-liquid’ compositions of the present invention are free from toxic antimicrobial preservatives that are usually, halogenated molecules or formaldehyde donors or parabens. The compositions either use food preservatives like sodium benzoate, sodium dehydroacetate or potassium sorbate or even without these giving the option of ‘preservative-free’ offering with N-acyl glycines.

The ‘powder-to-liquid’ compositions of the present invention are completely bio-degradable, and all ingredients are eco-friendly. The compositions are free from acrylates, EDTA, silicones, phthalates/BHT, phosphates etc. The ‘powder-to-liquid’ compositions of the present invention are based on eco-friendly and 100% biodegradable ingredients. The major portion (90%) of these compositions is made up of anionic and zwitterionic surfactants are bio-based, derived from vegetable oil. The combinations anionic surfactants are manufactured in an eco-friendly way meeting the principles of ‘green’ chemistry. And finally, the ‘powder’ compositions offered are concentrated that avoid water and other formulating aids, thus significantly reducing the amount of plastic in packaging. This is achieved without compromising the ease-of -use, performance and aesthetics.

### EXPERIMENTAL

### Powder Blends of Sodium O-Acyl Methyl Isethionate with Sodium Salt of N-Acylamino Acid Surfactants

Powder blends of sodium O-acyl methyl isethionate with sodium salt of N-acylamino acid surfactants (Table 1) are synthesized as per the procedure described in U.S. Pat. No. 9,308,156 B2 by Koshti et al. The aqueous solutions obtained as per the procedure given in U.S. Pat. No. 9,308,156 B2 are spray-dried to convert them into powder. Similarly,coco amidopropyl betaine (Galaxy CAPB) and lauramido propyl betaine (Galaxy LAPB) (both 36% solids) are procured from Galaxy Surfactants and spray-dried to convert them into powder form. N-Acyl amino acid surfactants used in the present invention are sodium lauroyl sarcosionate, sodium lauroyl glutamate, sodium lauroyl/cocoyltaurate and sodium lauroyl glycinate. O-Acyl methyl isethionate used are sodium lauroyl methyl isethionate and sodium cocoyl methyl isethionate.

Synthesis of a Blend of Sodium Lauroyl Methyl Taurate and Sodium Lauroyl Methyl Isethionate (1:1): TLMI 11 of Table 1

Lauroyl chloride used in this experiment had the following alkyl chain distribution: C₈: 0.5%, C₁₀: 0.5%, C₁₂: 99%.

To stirred lauroyl chloride (223 g, 1.00 gmol) under slow purging of nitrogen at room temperature, sodium methyl isethionate (85.5 g, 0.5 gmol) was added, and the slurry was stirred at 55-60° C. for 4 h. The HCl gas generated was absorbed in alkali solution and the progress of reaction was monitored by IR spectrum analysis. The FTIR spectrum of the intermediate showed the presence of unreacted lauroyl chloride (carbonyl stretch at 1800 cm⁻¹), sodium lauroyl methyl isethionate (carbonyl of ester at 1734 cm⁻¹) and disappearance of hydroxyl stretch (3323 cm⁻¹) of sodium methyl isethionate.

This fluid viscous reaction mass (285.5 g) was cooled to 35-40° C. and then added slowly to a stirred aqueous solution of sodium N-methyl taurate (217 g, 0.525 gmol) in water (682 g) along with sodium hydroxide solution (48.8%, 58.5 g, 0.7 gmol) simultaneously while maintaining the pH of the reaction mass between 10.5 to 11 and the temperature between 15 to 25° C. The addition was completed in two hours and the reaction mass was stirred for another 4 h at 25-30° C. The pH was adjusted to 7.5 with HCl. The solids content of the reaction mass was adjusted to 30% solids content to yield 1267 g of aqueous solution as final product.

IR spectrum of dried sample of TLMI 11 showed carbonyl stretch of amide at 1600-1620 cm⁻¹ and NH stretch at 3344 cm⁻¹. The other significant stretching frequencies were carbonyl of ester of alkanoyl isethionate at 1734 cm⁻¹ and total disappearance of carbonyl stretching frequency (1800 cm⁻¹) of lauroyl chloride. The analysis of the above aqueous surfactant blend and spray dried powder is given in Table 2.

### Synthesis of a Blend of Sodium Lauroyl Glutamate and Sodium Lauroyl Methyl Isethionate (1:1): GLMI 11 of Table 1

The lauroyl chloride used in this experiment had the following alkyl chain distribution: C₈: 0.5%, C₁₀: 0.5%, C₁₂: 99%.

To a stirred lauroyl chloride (223 g, 1.0 gmol) under slow purging of nitrogen at room temperature, sodium methyl isethionate (85.5 g, 0.5 gmol) was added and the slurry was stirred at 60-65° C. for 4 h. The HCl gas generated was absorbed in alkali solution and the progress of reaction was monitored by IR spectrum analysis. The FTIR spectrum of the intermediate showed the presence of unreacted lauroyl chloride (carbonyl stretch at 1800 cm⁻¹), sodium lauroyl methyl isethionate (carbonyl of ester at 1734 cm⁻¹) and disappearance of hydroxyl stretch (3323 cm⁻¹) of sodium methyl isethionate.

This fluid viscous reaction mass (285.5 g) was cooled to 35-40° C. and then added slowly to a stirred aqueous solution of sodium mono glutamate (98 g, 525 gmol) in water (751.5 g) along with sodium hydroxide solution (48.8%, 92 g, 1.1025 gmol) simultaneously while maintaining the pH of the reaction mass between 10.5 to 11.0 and the temperature between 15 to 25° C. The addition was completed in two hours and the reaction mass was stirred for another 4 h at room temperature of 25-30° C. The pH was adjusted to 7.5 with HCl. The solids content of the reaction mass adjusted to 33% solids content to yield 1268 g of aqueous solution product.

IR spectrum of dried sample showed carbonyl stretch of amide at 1600-1620 cm⁻¹ and NH stretch at 3344 cm⁻¹. The other significant stretching frequencies were carbonyl of ester of alkanoyl isethionate at 1734 cm⁻¹ and total disappearance of carbonyl stretching frequency (1800 cm⁻¹) of lauroyl chloride. The analysis of the above aqueous surfactant blend and spray dried powder is given in Table 2.

### Synthesis of a Blend of Sodium Lauroyl Methyl Taurate and Sodium Lauroyl Methyl Isethionate (2:1): TLMI 21 of Table 1

Lauroyl chloride used in this experiment had the following alkyl chain distribution: C₈: 0.5%, C₁₀: 0.5%, C₁₂: 99%.

To a stirred lauroyl chloride (335 g, 1.5 gmol) under slow purging of nitrogen at room temperature, sodium methyl isethionate (85.5 g, 0.5 gmol) was added, and the slurry was stirred at 60-65° C. for 4 h. The HCl gas generated was absorbed in alkali solution and the progress of reaction was monitored by IR spectrum analysis. The FTIR spectrum of the intermediate showed the presence of unreacted lauroyl chloride (carbonyl stretch at 1800 cm⁻¹), sodium lauroyl methyl isethionate (carbonyl of ester at 1734 cm⁻¹) and disappearance of hydroxyl stretch (3323 cm⁻¹) of sodium methyl isethionate.

This fluid viscous reaction mass (395 g) was cooled to 35-40° C. and then added slowly to a stirred aqueous solution of sodium methyl taurate (433.5 g, 1.05 gmol) in water (988 g) along with sodium hydroxide solution (48.8%, 110 g, 1.3125 gmol) simultaneously while maintaining the pH of the reaction mass between 10.5 to 11.0 and the temperature between 15 to 25° C. The addition was completed in two hours and the reaction mass was stirred for another 4 h at 25-30° C. The pH was adjusted to 7.5 with HCl. The solids content of the reaction mass adjusted to 30% solids content to yield 1955 g of aqueous solution product.

IR spectrum of dried sample showed carbonyl stretch of amide at 1600-1620 cm⁻¹ and NH stretch at 3344 cm⁻¹. The other significant stretching frequencies were carbonyl of ester of alkanoyl isethionate at 1734 cm⁻¹ and total disappearance of carbonyl stretching frequency (1800 cm⁻¹) of lauroyl chloride. The analysis of the above aqueous surfactant blend and spray dried powder is given in Table 2.

### Synthesis of a Blend of Sodium Cocoyl Methyl Taurate and Sodium Cocoyl Methyl Isethionate (1:1): TCMI 11 of Table 1

The cocoyl chloride used in this experiment had the following alkyl chain distribution: C₈: 6%, C₁₀: 6%, C₁₂: 61%, C₁₄: 22.5%, C₁₆: 4%, C₁₈: 0.5%.

To a stirred cocoyl chloride (223 g, 1.0 gmol) under slow purging of nitrogen at room temperature, sodium methyl isethionate (85.5 g, 0.5 gmol) was added and the slurry was stirred at 60-65° C. for 4 h. The HCl gas generated was absorbed in alkali solution and the progress of reaction was monitored by IR spectrum analysis. The FTIR spectrum of the intermediate showed the presence of unreacted cocoyl chloride (carbonyl stretch at 1800 cm⁻¹), sodium cocoyl methyl isethionate (carbonyl of ester at 1734 cm⁻¹) and disappearance of hydroxyl stretch (3323 cm⁻¹) of sodium methyl isethionate.

This fluid viscous reaction mass (286 g) was cooled to 35-40° C. and then added slowly to a stirred aqueous solution of sodium methyl taurate (217 g, 0.525 gmol) in water (681 g) along with sodium hydroxide solution (48.8%, 56 g, 0.67 gmol) simultaneously while maintaining the pH of the reaction mass between 10.5 to 11.0 and the temperature between 15 to 25° C. The addition was completed in two hours and the reaction mass was stirred for another 4 h at room temperature of 25-30° C. The pH was adjusted to 7.5 with HCl. The solids content of the reaction mass adjusted to 30% solids content to yield 1262 g of aqueous solution product.

IR spectrum of dried sample showed carbonyl stretch of amide at 1600-1620 cm⁻¹ and NH stretch at 3344 cm⁻¹. The other significant stretching frequencies were carbonyl of ester of alkanoyl isethionate at 1734 cm⁻¹ and total disappearance of carbonyl stretching frequency (1800 cm⁻¹) of cocoyl chloride. The analysis of the above aqueous surfactant blend and spray dried powder is given in table 2.

Other blends of acyl methyl isethionate and acyl amino acids like TLMI 41, TLMI 14, GLMI 41 and GLMI 14 are synthesized by similar process.

TABLE 2
Analysis of blends of O-acyl methyl isethionate and N-acyl
glutamate blends prepared by pseudo one pot synthesis.

| | TLMI | GLMI | TLMI | TCMI | TLMI | TLMI | GLMI | GLMI |
|---|---|---|---|---|---|---|---|---|
| Parameters | 11 | 11 | 21 | 11 | 41 | 14 | 41 | 14 |
| Appearance | clear | clear | clear | clear | clear | clear | clear | clear |
| | liquid | liquid | liquid | liquid | liquid | liquid | liquid | liquid |
| Viscosity at | 3050 cps | 400 cps | 2600 cps | 900 cps | 425 cps | 1540 cps | <100 cps | 3050 cps |
| 25° C. | | | | | | | | |
| pH as such | 7.6 | 7.75 | 7.65 | 7.70 | 7.25 | 7.30 | 7.50 | 7.35 |
| Sodium | 3.90 | 3.80 | 3.98 | 3.79 | 4.10 | 4.05 | 4.30 | 2.80 |
| chloride, | | | | | | | | |
| (% w/w) | | | | | | | | |
| Total solids | 31.25 | 33.35 | 31.25 | 31.38 | 31.80 | 31.65 | 30.10 | 29.45 |
| (% w/w) | | | | | | | | |
| Active | 27.35 | 29.55 | 27.27 | 27.59 | 27.70 | 27.60 | 25.8 | 26.65 |
| matter* | | | | | | | | |
| (% w/w) | | | | | | | | |

Analysis after spray drying:

| | | | | | | | | |
|---|---|---|---|---|---|---|---|---|
| Moisture | 2.60 | 3.30 | 2.75 | 2.88 | 2.60 | 2.85 | 2.75 | 2.88 |
| content (% | | | | | | | | |
| by mass) | | | | | | | | |
| Active | 85.3 | 85.4 | 85.05 | 85.32 | 84.88 | 84.75 | 83.45 | 87.92 |
| matter**(% | | | | | | | | |
| w/w) | | | | | | | | |
| Sodium | 12.10 | 11.30 | 12.20 | 11.80 | 12.52 | 12.40 | 13.80 | 9.20 |
| chloride (% | | | | | | | | |
| w/w) | | | | | | | | |

*solids content - sodium chloride
**100 - sod. chloride content - moisture content

The present invention is now illustrated by way of non-limiting examples.

### EXAMPLES

### Comparative Examples 1 and 2 of U.S. Pat. No. 11,045,404

Disodium cocoyl glutamate, disodium lauroyl glutamate, cocamidopropyl betaine, lauramidopropyl hydroxy sultaine and cocamidopropyl hydroxy sultaine are procured from Galaxy surfactants ltd. These surfactants are deployed in formulation as directed in U.S. Pat. No. 11,045,404.

The comparative examples 1 and 2 are prepared according to the disclosure of US'404 (Ex. 1 of Table 2 and Ex. 10 of Table 7 respectively) using same amount of disodium cocoyl glutamate and lauramidopropyl hydroxy sultaine and at the reported pH of 5.3. For comparative example 1 it is possible to get a composition with viscosity number of 2820 cps. But at pH 5.4 the viscosity drops to 820 cps and it further loses the viscosity to be water-thin at pH 5.5 (normal human skin pH). Similarly, at pH 5.1, the viscosity drops to 1000 cps. (Comparative Example 1).

TABLE 3a
Comparative Example 1 (Example 1 of Table 2 of US'404)

| Ingredients | Comp. Example 1 |
|---|---|
| Disodium cocoyl glutamate | 28 |
| Lauramidopropyl hydroxy sultaine | 21.27 |
| Water | make up 100 |
| pH (citric acid) | 5.2 |
| Solid's content | 19.5 |
| Anionic surfactant to zwitterionic surfactant ratio | 1:1.2 |
| Viscosity at pH (5.2) | 2800 cps |
| Viscosity at pH (5.1) | 1300 cps |
| Viscosity at pH (5.4) | 280 cps |
| Viscosity at pH (5.5) | <100 cps |
| Viscosity at pH (4.5) | 200 cps |

Comparative example 2, prepared according to the disclosure of US'404 (Ex. 10, Table 7), contains cocoyl glutamate with cocoamidopropyl betaine which results in viscous but turbid composition with viscosity of 4300 cps at pH of 5.1. Lowering pH to 5.0 increases the opaqueness significantly with increase in viscosity to 16,400 cps. Further lowering of the pH by just 0.2 units, viscosity drops to 3150 cps and opaqueness is intensified almost giving appearance of precipitation of surfactant. This illustrates the unpredictable arrangement of surfactant aggregates that would be challenging to reproduce consistently on a commercial scale for industrial applicability. In this example too, the viscosity at skin pH of 5.5 is water-thin i.e., less than 100 cps. Hence, cocoyl glutamates and zwitterionic surfactants, depending upon the concentration of surfactants and the pH exhibit a random thickening point that is often times nowhere near the desired pH value and secondly, the narrow window of this thickening point is very difficult to achieve consistently in large scale manufacture.

TABLE 3b
Comparative Example 2 (Example 10 of Table 7 of US'404)

| Ingredients | Comp. Example 2 |
|---|---|
| Disodium cocoyl glutamate | 25 |
| Cocoamidopropyl betaine | 20 |
| Water | make up 100 |
| Citric acid for pH adjustment | 5.2 |
| Solid's content | 15.5 |
| Anionic surfactant to zwitterionic surfactant ratio | 1:1 |
| Viscosity at pH (5.2) | 4000 cps, hazy |
| Viscosity at pH (5.3) | 260 cps |
| Viscosity at pH (5.4) | <100 cps |
| Viscosity at pH (5.5) | <100 cps |
| Viscosity at pH (5.0) | 16,400 cps, hazy |
| Viscosity at pH (4.8) | 3150 cps, hazy |

### Examples 1 to 22: Preparation of the ‘Powder-to-Liquid’ Wash Formulations Using Surfactant Blends of Table 1, Alkylamidopropyl Betaines and Other Additives

The anionic surfactants of Table 1 and alkylamidopropyl betaines are spray dried separately using laboratory glass spray dryer (Model GLS 100, Hemraj Engineering, Mumbai, India), with drying capacity of 500 mL of liquid per hour. The aqueous surfactant solutions (30-40% solids content) are fed at ambient temperature and is spray dried with air at 125 to 130° C.

The active content of powder betaines is around 85% (Active content =solids content — sodium chloride content—moisture content). Rest of the ingredients of Table 4 and Table 4a in powder form are considered as 100% active.

Oil-soluble fragrance Sonarex 9090409, is procured from Sonarex High Tech Aromatics Pvt Ltd, India, whereas water-soluble fragrance, CPL AR 634A412 is procured from CPL Aromas, Mumbai.

All ingredients (Table 4) in powder form are blended together to get free-flowing powders of bulk density varying from 0.4 to 0.5 gm per cm3 and with all particles with size of less than 300 micron.

Galguard LipoG as a blend of capryloyl glycine and undecylenoyl glycine in the molar ratio of 1:1. It is commercially offered (Galaxy Surfactants Ltd, India) as flakes which are pulverized to make it suitable for dry mixing. GHTC (Guar hydroxy propyl trimethyl ammonium chloride), PCA-Na (Pyrrolidine carboxylic acid, sodium salt), and sodium gluconate are available in fine powder form. Crystalline benzoic acid and citric acid are pulverized before using them in blend. Sodium chloride is used without any particle reduction.

The ‘liquid’ wash compositions are prepared by dissolving (diluting) these powder blends (Table 4) in water at around 16 to 22 solids, % w/w (4 to 5 times dilution) by gentle agitation at ambient temperature. The liquid compositions (300 mL) are made in 500 mL measuring cylinders with stoppers. The weighed quantity of powder is transferred to a stoppered cylinder and the requisite amount of tap water is added and gently shaken (vertical shakes) with the stopper on. The liquid is allowed to sit in the glass cylinder for a while (5 mins) before taking it for analysis. The resultant liquid personal care compositions are transparent and with pH close to skin pH. Examples 17, 18, and 19 of Table 4a on the other hand (where the ratio of O-acyl methyl isethionate and N-acyl amino acid surfactants is varied from 4:1 to 1:4) results in poor viscosity even at high solids content of 22%. It is believed that such poor viscosity results in difficulty in handling by the end consumer. The reconstitutable concentrated powder composition of the present invention when diluted by 4 to 12 times by weight of said powder, with water affords a transparent liquid formulation with pH ranging from 5.0 to 6.0 and viscosity ranging from 1,000 to 15,000 cps at 25° C. and such viscosity ensure eases in handling in large scale production as well as for end consumers. The reconstituted formulation of present invention has pH of between 5 to 6 (skin pH). Also, the reconstituted formulation of present invention did not result in fall of viscosity within desired pH range. Further, getting the desired viscosity without using any thickening agent is the major advantage of the invention.

TABLE 4
Concentrated powder blends and analysis of reconstituted formulations.

| | Ex | Ex | Ex | Ex | Ex | Ex | Ex | Ex |
|---|---|---|---|---|---|---|---|---|
| Ingredients in | 1 | 2 | 3 | 4 | 5 | 6 | 7 | 8 |
| powder from | Quantities in % w/w | | | | | | | |
| GLMI 11 | 60 | 60 | — | — | — | — | — | — |
| GLMI 21 | — | — | 60 | — | — | — | — | — |
| TLMI 11 | — | — | — | 60 | 60 | 60 | 60 | — |
| TLMI 21 | — | — | — | — | — | — | — | — |
| TCMI 11 | — | — | — | — | — | — | — | 60 |
| CAPB | 30 | — | 30 | 30 | | 35.5 | 34.5 | 30.8 |
| LAPB | — | 30 | — | — | 34 | — | — | — |
| Benzoic acid | 3.5 | 3.5 | 3.5 | 4.0 | — | — | — | 3.5 |
| Galguard | — | — | — | — | 2.5 | 2.5 | 2.5 | — |
| LipoG | | | | | | | | |
| GHTC | — | — | — | — | — | — | — | — |
| BAPDA | — | — | — | — | — | — | — | — |
| Citric acid | 1.4 | 1.4 | 1.4 | 0.7 | 0.5 | 0.5 | 0.5 | 0.6 |
| Sodium | 4.5 | 4.5 | 4.5 | 4.5 | 2.5 | 1.0 | 2.0 | 4.5 |
| chloride | | | | | | | | |
| Sodium | 0.6 | 0.6 | 0.6 | 0.8 | 0.5 | 0.5 | 0.5 | 0.6 |
| Gluconate | | | | | | | | |
| PCA-Na | — | — | — | — | — | — | — | — |
| CPL | — | — | — | — | — | — | — | — |
| Aromas | | | | | | | | |
| AR | | | | | | | | |
| 634412 | | | | | | | | |
| Sonarex | — | — | — | — | — | — | — | — |
| 9090409 | | | | | | | | |

Formulations Analysis after reconstitution of above powders

| | | | | | | | | |
|---|---|---|---|---|---|---|---|---|
| Appearance | Clear | Clear | Clear | Clear | Clear | Clear | Clear | Clear |
| of solution | | | | | | | | |
| Color, | <100 | <100 | <100 | <100 | <100 | <100 | <100 | <100 |
| APHA | | | | | | | | |
| Solids | ~16.5 | ~16.5 | 16.4 | ~16.5 | 21.2 | 21 | 18.5 | 16.4 |
| content | | | | | | | | |
| pH | 5.4 | 5.3 | 5.3 | 5.4 | 5.35 | 5.4 | 5.3 | 5.3 |
| Viscosity | 3000 | 2700 | 2150 | 2450 | 6500 | 4000 | 2100 | 1150 |
| (cps at | | | | | | | | |
| 25° C.) | | | | | | | | |

| | Ex | Ex | Ex | Ex | Ex | Ex | Ex | Ex | Ex |
|---|---|---|---|---|---|---|---|---|---|
| Ingredients in | 9 | 10 | 11 | 12 | 13 | 14 | 15 | 16 | 16a |
| powder from | Quantities in % w/w | | | | | | | | |
| GLMI 11 | — | — | — | — | — | — | — | — | — |
| GLMI 21 | — | — | 66 | — | 60 | 60 | — | — | — |
| TLMI 11 | — | — | — | 60.5 | — | — | — | — | 58 |
| TLMI 21 | 67 | — | — | — | — | — | 61 | 61 | — |
| TCMI 11 | — | 67 | — | — | — | — | — | — | — |
| CAPB | 28 | 28 | 28 | 31 | 30 | — | — | — | 28.5 |
| LAPB | — | — | — | — | — | 30 | 31 | 30 | — |
| Benzoic acid | 3.0 | 3.0 | 3.0 | 3.0 | 3.0 | — | — | — | 3.0 |
| Galguard LipoG | — | — | — | — | — | 3.0 | 3.0 | 3.0 | — |
| GHTC | — | — | — | — | — | — | — | 1.0 | — |
| BAPDA | — | — | — | — | — | — | — | — | 3.5 |
| Citric acid | 0.5 | 0.5 | 1.5 | | 2.0 | 2.0 | — | — | 1.5 |
| Sodium | 1.0 | 1.0 | 1.0 | 3.0 | 3.0 | 3.0 | 3.0 | 3.0 | 3.0 |
| chloride | | | | | | | | | |
| Sodium | 0.5 | 0.5 | 0.5 | 0.5 | 0.5 | 0.5 | 0.5 | 0.5 | 1.0 |
| Gluconate | | | | | | | | | |
| PCA-Na | — | — | — | 0.5 | — | — | — | — | — |
| CPL Aromas | — | — | — | 1.5 | 1.5 | — | — | 1.5 | 1.5 |
| AR 634412 | | | | | | | | | |
| Sonarex | — | — | — | — | — | 1.5 | 1.5 | — | — |
| 9090409 | | | | | | | | | |

Formulations Analysis after reconstitution of above powders

| | | | | | | | | | |
|---|---|---|---|---|---|---|---|---|---|
| Appearance of | Clear | Clear | Clear | Clear | Clear | Clear | Clear | Clear | Clear |
| solution | | | | | | | | | |
| Color, APHA | <100 | <100 | <100 | <100 | <100 | <100 | <100 | <100 | <100 |
| Solids content | 21.0 | 21.0 | 21.0 | 16.35 | 16.65 | 16.65 | 16.35 | 16.5 | 16.6 |
| pH | 5.4 | 5.4 | 5.4 | 5.3 | 5.4 | 5.4 | 5.4 | 5.8 | 5.4 |
| Viscosity (cps | 10000 | 6050 | 14500 | 7300 | 6050 | 5440 | 8850 | 6400 | 2470 |
| at 25° C.) | | | | | | | | | |

Examples 17 to 22 of table 4a demonstrate the concentrated powder blends of O-acyl methyl isethionate and amino acid surfactants with ratio of 4:1 and 1:4. The compositions on reconstitution with water results into viscosity of less than 100 cps.

TABLE 4a
Reconstitutable powder compositions with O-acyl methyl isethionate
and amino acid surfactants at ratio of 4:1 and 1:4

| | Ex | Ex | Ex | Ex | Ex | Ex |
|---|---|---|---|---|---|---|
| Ingredients in | 17 | 18 | 19 | 20 | 21 | 22 |
| powder from | Quantities in % w/w | | | | | |
| GLMI 41 | — | — | — | 54 | — | — |
| GLMI 14 | — | — | | — | 55 | 55 |
| TLMI 41 | 55 | 54.5 | — | — | — | — |
| TLMI 14 | | — | 54 | — | — | — |
| CAPB | 35 | 35 | 35 | 34 | 35 | 35 |
| Benzoic acid | 4.5 | 4.5. | 4.5 | 4.5 | 4.5 | 4.5 |
| Galguard | — | — | — | — | — | — |
| LipoG | | | | | | |
| Citric acid | | 0.5 | 1.0 | 2.0 | — | — |
| Sodium | 3.0 | 3.0 | 3.0 | 3.0 | 3.0 | 3.0 |
| chloride | | | | | | |
| Sodium | 1.0 | 1.0 | 1.0 | 1.0 | 1.0 | 1.0 |
| Gluconate | | | | | | |
| CPL Aromas | 1.5 | 1.5 | 1.5 | 1.5 | 1.5 | 1.5 |
| AR 634412 | | | | | | |

Formulations Analysis after reconstitution of above powders

| | | | | | | |
|---|---|---|---|---|---|---|
| Appearance of | Clear | Clear | Clear | Clear | Clear | Clear |
| solution | | | | | | |
| Color, APHA | <100 | <100 | <100 | <100 | <100 | <100 |
| Solids content | 10.8 | 21.7 | 21.65 | 10.60 | 10.82 | 21.75 |
| pH | 5.25 | 5.4 | 5.45 | 5.30 | 5.25 | 5.45 |
| Viscosity (cps | <100 | 820 | 140 | 560 | <100 | 730 |
| at 25° C.) | | | | | | |

1. A reconstitutable concentrated powder composition, said composition comprising:
a. 50 to 70% by weight of total powder composition, a blend of O-acyl methyl isethionate and N-acyl amino acid surfactants;
b. 25 to 40% by weight of total powder composition, an alkylamidopropyl betaine;
c. 2.0 to 6.0% by weight of total powder composition, an antimicrobial preservative; and
d. optionally, 1.0 to 10.0% by weight of total powder composition, one or more cosmetically or pharmaceutically acceptable auxiliary,
wherein said blend of O-acyl methyl isethionate and N-acyl amino acid surfactants is prepared sequentially from same fatty acid chloride; said O-acyl methyl isethionate and N-acyl amino acid surfactants being present at a ratio ranging from 2:1 to 1:2; and said reconstitutable concentrated powder composition when diluted with water by 4 to 12 times by weight of said powder, affords a transparent liquid formulation with pH ranging from 5.0 to 6.0 and viscosity ranging from 1,000 to 15,000 cps at 25° C.

2. The reconstitutable concentrated powder composition as claimed in claim 1, wherein O-acyl methyl isethionate surfactant is selected from sodium lauroyl methyl isethionate, sodium cocoyl methyl isethionate and mixtures thereof.

3. The reconstitutable concentrated powder composition as claimed in claim 1, wherein N-acyl amino acid surfactant is selected from sodium lauroyl glutamate, sodium cocyl glutamate, sodium lauroyl methyl taurate, sodium cocoyl methyl taurate, sodium lauroyl sarcosinate, sodium lauroyl glycinate and mixtures thereof.

4. The reconstitutable concentrated powder compositions as claimed in claim 1, wherein alkylamidopropyl betaine is selected from coco amidopropyl and lauramido propyl betaine.

5. The reconstitutable concentrated powder composition as claimed in claim 1, wherein antimicrobial preservative is selected from one or more of benzoic acid, sorbic acid, dehydroacetic acid, undecylenoyl glycine, capryloyl glycine and mixtures thereof.

6. The reconstitutable concentrated powder composition as claimed in claim 1, wherein the cosmetically or pharmaceutically acceptable auxiliaries are selected from one or more hair care actives, skin care actives, water-soluble dry herbal extracts, vitamins, chelating agents, inorganic salts, moisturizing agents, soothing agents, nourishing agents, conditioners, colors and fragrances.

7. The reconstitutable concentrated powder composition as claimed in claim 1, wherein the composition is formulated as a handwash, facewash, bodywash or a shampoo.
